(19) United States
(12) Patent Application Publication
(10) Pub. No.: US 2017/0232026 A1
(43) Pub. Date: Aug. 17, 2017

(54) PHARMACEUTICAL COMPOSITION IN IVERMECTIN EMULGEL FOR VETERINARY USE AS A PROMOTER SYSTEM AND BIO-ADHESIVE IN ANTIPARASITIC TREATMENT, AND METHOD FOR THE PRODUCTION THEREOF

(71) Applicant: UNIVERSIDAD NACIONAL AUTONOMA DE MEXICO, Mexico City (MX)

(72) Inventors: David QUINTANAR GUERRERO, Cuautitlan Izcalli (MX); Juan Pablo MARTINEZ LABAT, Tepotzotlan (MX); Susana Elisa MENDOZA ELVIRA, Coacalco (MX); Gustavo VIDAL ROMERO, Nezahualcoyotl (MX); Victor Andres TRENADO HERNANDEZ, Cuautitlan Izcalli (MX)

(21) Appl. No.: 15/503,033

(22) PCT Filed: Aug. 12, 2015
(86) PCT No.: PCT/MX2015/000114

§ 371 Date: Feb. 10, 2017

(30) Foreign Application Priority Data
Aug. 12, 2014 (MX) MX/A/2014/009688

(51) Int. Cl. A61K 31/7048; A61K 47/32; A61K 47/26; A61K 47/44; A61K 47/18; A61K 9/06; A61K 47/10

(52) U.S. Cl. CPC A61K 31/7048; A61K 9/06; A61K 47/32; A61K 47/10; A61K 47/44; A61K 47/18; A61K 47/26

(57) ABSTRACT

The invention relates to a pharmaceutical composition comprising: at least one antiparasitic active ingredient for providing the pharmacological therapeutic effect; an agent having bio-adhesive properties that helps to reduce the motility and reproduction of the parasites, increasing the period of time for which the product remains on the skin of the animals; an agent having permeation- or absorption-promoting properties that contributes to increasing the cutaneous permeability of the active ingredient; a surfactant agent that is absorbed in an oil-water interface, and as a result, the molecules of said surfactant agent form a kind of bridge between the polar phase (water) and the non-polar phase (oil), thereby making the transition between both phases less abrupt; an oil which will form the oily phase of the emulsion, and which will incorporate the active ingredient; and a neutralizing agent.

### FIELD OF INVENTION

Present invention is related to principles and techniques used in veterinary pharmaceutical industry for antiparasitic treatment in small and large species and more specifically, it relates to an ivermectin emulgel pharmaceutical composition for veterinary use as bioadhesive promoter system for percutaneous application, being a new alternative for antiparasitic treatment; the invention is also related to a method for obtaining said composition.

### BACKGROUND OF INVENTION

Ivermectin is a modified form of a macrocyclic compound called avermectin derived from Streptomyces avermitilis fungus. It shows a similar structure to macrolide antibiotics but lacks antibacterial activity. However, it is very active against a wide variety of parasites that infect animals and humans, including nematodes, mites and insects where gamma amino butyric acid neurotransmitter is inhibited leading to flaccid paralysis, therefore having anthelmintic properties.

Fermentation products of Streptomyces avermitilis actinomycetes correspond to four pairs of related homologous compounds wherein avermectin B1a is the most important as produced in largest amount and where ivermectin semisynthetic derivative has been obtained therefrom. Ivermectin is 22, 23-dihydroavermectin B1a, a crystalline to yellowish white powder, insoluble in water but soluble in methanol and ethanol at 95%. Ivermectin possesses a wide activity spectrum against adult and larvae status of parasitic nematodes and arthropods in domesticated animals and its action mode makes it fully active against parasites resistant to other anthelmintics.

It was originally thought that macrolide endectocides increased gamma-amino butyric acid (GABA) release from nervous system synaptosomes which in turn allowed activation of chloride channels.

Nowadays is known that these compounds activate glutamate-activated chloride channels in invertebrates by means of a specific binding site and affecting motility and pharingeal pumping in parasitic nematodes. Action mode of avermectins is a selective parasite paralysis by increasing muscle permeability to chloride ions. Avermectins enhance glutamate effects at low concentrations and directly open glutamate-activated channels at high concentrations. The drug has few effects in adult parasites but affects larvae under development and blocks micropyle output from adult vermes uterus.

The selective therapeutic effect of avermectins may be explained by an action over glutamate-chloride channels which are present in parasite nematodes but not in host animal, which in addition to the fact that GABA-mediated neurotransmission in mammals is limited to central nervous system and since endectocide macrolides do not easily traverse blood-brain barrier, these anthelmintics thus explain their high safety margin.

Drug anthelmintic activity over parasite is related to the presence of effective concentrations in terms of level and duration at action site.

Macrocyclic lactones are characterized by possessing a persistent anthelmintic effect against gastrointestinal nematodes in ruminants and by a powerful antiparasitic activity at very low doses against pathogen nematodes and ectoparasites in domesticated animals. Within above group, ivermectin possess a wide activity spectrum against many domesticated animal ectoparasites.

Pharmacokinetics and activity of avermectins and milbemicines are particularly influenced by physicochemical properties of active molecules. It is known that excipient significantly influences speed and duration of drug absorption process and may modify the plasma concentration profile.

Route of administration of anthelmintic drugs may affect their distribution, bioavailability and persistence of their concentrations in the body. Ivermectin is a highly lipophilic drug which is suitably absorbed when administered whether parenterally, topically or orally, being widely distributed in the body. It is concentrated in adipose tissue where is slowly released for transforming a further fraction thereof in less liposoluble metabolites.

Pour-on cutaneous applications consist of spilling the product over animal dorsal midline, being distributed along the body and reaching the places where parasites are harbored. This technique is recommended to be applied in places where it is not easy to handle animals as it offers several advantages: it may be adhered on animal body surface, secretions from sudoriferous and sebaceous glands form an emulsion acting as vehicle to increase dermal distribution of active substance, the formulations act over parasites by contact and by means of emanated vapors, forming an atmosphere surrounding treated animal body. Concentration of active substance in a pour-on is considerably higher than a spray or implant. Many animals secrete oily tallow which is necessary for a formulation containing an organic solvent, a surfactant and/or an absorption agent unless active substance is tallow soluble or miscible. Tallow starts emulsification with sweat until the follicle pore is full as well as animal skin and the sections below the hair.

The purpose of formulating a drug within a veterinary controlled release dosage form is for producing a product where drug is released in a predetermined time and meets the veterinary therapeutic conditions. Controlled release veterinary systems are out of necessity innovative in their design and formulae due to drug release requirements or to the environment where it will be located. Compared to controlled release products in humans, veterinary products are designed to release the drug through very long periods (weeks or months compared to hours or days). Veterinary release systems are usually manufactured with polymers which present a biocompatibility history, they are biologically inert, having regulatory approval and not being expensive; such polymers may be biodegradable or non-biodegradable. Semisolid pharmaceutical preparations may be defined as a product expected for application on skin or mucous membranes and sometimes having systemic effect from application site. Semisolid dosages are generally complex formulations having complex structural elements.

Oil-in-water and water-in-oil emulsions are widely used because of their therapeutic properties and as vehicles for delivery of several drugs into skin. Emulsions have certain degree of elegance and they are easy to wash whenever is desired. They have also a great capability to penetrate skin. Further, the formulator may control viscosity, appearance and degree of greasiness of cosmetic or dermatologic emulsions. Oil-in-water emulsions are more used as base of easily washable drugs and generally with cosmetic purpose, while water-in-oil emulsions are widely used for dry skin treatments and emollient applications.

Gels are semisolid systems containing a cross-linked polymeric matrix (whether natural or synthetic gums) and organized in three dimensions suspended in a liquid, retained through addition of a gelling agent. They generally contain a low polymer concentration (less than 10%, usually between 0.5 and 2%, depending on the polymer). Gels are an intermediate state of matter containing both solid and liquid components.

USP defines gels as a semisolid system composed by small inorganic particles or by large organic molecules internally penetrated by a liquid.

In the Pharmacopea of the Mexican United States (FEUM by its Spanish acronym), a gel is defined as a semisolid preparation comprising the active principle(s) generally constituted by macromolecules dispersed in a liquid which may be water, alcohol or oil, forming a network which traps the liquid and restricts its movement, therefore they are viscous preparations.

Those are usually attractive for consumers as they show a clean, pure and natural image, providing a fresh, non-fatty sensation, allowing contact of active ingredients with skin without obstruction and having good thickening and efficient suspension of insoluble particles.

Thus, emulgels are emulsions whether oil-in-water or water-in-oil type, which are gelled by a combination with a gelling agent. They have high patient acceptance as they possess above mentioned advantages of emulsions and gels. Therefore, they have been used recently as vehicles for administering several drugs into skin.

There is a large variety of documents related with the matter of present invention within the state of the art, such as international publication No. WO/2009/070687 referred to pharmaceutical and veterinary formulations offering higher solvency and stability for pharmaceutical and veterinary agents administered to animals, especially ruminants. Moreover, the invention refers to pour-on formulations for fighting parasites in animals such as cows and sheep. In some embodiments the invention provides glycol-ether based pour-on formulations comprising a composition comprising a fasciola drug such as for example, clorsulon (4-amino-6-trichloroethenyl-1, 3-benzene disulfonamide) and/or a macrolide or antiparasitic anthelmintic agent. In other embodiments, the invention provides pour-on formulations comprising at least an active agent, glycol ether and a stability enhancer. This invention also provides methods for eradication, control, and/or prevention of parasitic infestation in animals, such as cows and sheep.

Applications and combinations of ivermectin with other antiparasitic agents are described in Mexican patent application No. MX/a/2010/004852, where compositions for parasite control are disclosed; comprising a combination of ivermectin, abamectin, albendazol sulfoxide and triclorfon and the use of such compositions in preparation of a medicament for endoparasitic control.

U.S. Pat. No. 6,482,425 refers to a method and composition against parasites and particularly, ectoparasites, and also preferably small mammal endoparasites, particularly in dogs and cats. The composition contains a compound (A) of formula (I) and a compound (B) consisting of a macrocyclic lactone endectoparasiticide. From that above, it may be seen that said patent describes a formulation which combines parasitic compounds intending to enhance the therapeutic effect and its application is limited to small species such as dogs, cats and some birds, in addition to obtain a liquid system for located (spot-on) topical application.

U.S. Pat. No. 6,991,801 provides topical veterinary formulations such as spot-on and pour-on formulations which are used in treatment, control and prevention of infections by endo- and ectoparasites in warm-blood animals or birds such as horses, cattle, sheep, hogs and domesticated animals. This invention further provides a method to increase bioavailability of an anthelmintic agent contained in the formulation, which is susceptible to first-step metabolism in a warm-blooded animal or bird. Topical formulations of the invention comprise for instance, at least a macrolide anthelmintic compound and a second anthelmintic agent for instance, praziquantel, morantel and/or pirantel, a non-aqueous solvent which dissolves both the first anthelmintic agent and the macrolide anthelmintic compound and a thickening agent. Topical formulations of the invention containing praziquantel and/or morantel as the second anthelmintic agent exhibit improved systemic bioavailability by elimination of first-step metabolism of this compound and stability to exposure hydrolysis.

U.S. Pat. No. 7,754,696 describes an invermectin-in-water taste-pleasant, stable solution during massive medication of animals. Present formulation does not require the use of benzyl alcohol and is indefinitely stable in concentrated form and during a maximum term of 30 days when mixed with water.

U.S. Pat. No. 8,119,150 provides a safe and efficient insecticide composition suitable for treatment of a subject infested with an anthropoid parasite or to prevent infestation by an arthropod. The insecticide composition is a foamable composition including a first insecticide; at least an organic carrier selected from an organic hydrophobic carrier, a polar solvent, an emollient and mixtures thereof, in a concentration from about 2% to about 5%, or about 5% to about 10%, or about 10% to about 20%, or about 20% to about 50% by weight; about 0.1% to about 5% by weight of a surface-active agent; about 0.01% to about 5% by weight of at least a polymeric agent selected from a bioadhesive agent, a gelling agent, a film-forming agent and a phase-change agent, and (5) a liquefied or compressed propellant gas at a concentration of about 3% to about 25% by weight of total composition. However, as shown above, the patent does not show any relationship with the matter of present invention, as that is a foaming system comprising an insecticide directed to elimination and prevention of arthropod parasites such as spiders, lice and other insects. Moreover, it is proposed for human use and therefore it is not the same field of application.

U.S. patent application serial No. US20050191343 refers to revert micellar formulations for hydrophobic or lipophillic compounds, particularly therapeutic compounds.

An article titled “Effect of diethylenglycol-monoethyl ether as ivermectin topical release carrier.” Yazdanian, M. is also within the state of the art. The effect of diethylene glycol monoethyl ether (DGME; Transcutol) over ivermectin permeation, a wide-spectrum antiparasitic agent, through bovine skin was assessed through in-vitro permeation experiments, followed by serial subsections into skin to assess the ivermectin amount retained in skin. Ivermectin permeation through bovine skin was carried out by DGME and this improvement was dependent on DGME concentration. Ivermectin permeation was effectively improved in carriers with low DGME ratios but permeation improvement magnitude decreased as DGME ratio increased. Permeation was accompanied by formation of ivermectin cutaneous accumulations. Further, data reported that ivermectin cutaneous accumulation and flow were sensitive to DGME concentration gradient through skin. This fact suggested that ivermectin is impregnated with DGME, where it is very soluble. Therefore the improvement mechanism involves ivermectin solubilization by DGME and transport of DGME by itself through the skin. Based on these results, DGME seems to be a potential carrier for topical ivermectin administration by transportation through skin and through formation of cutaneous ivermectin accumulations.

As being apparent from above description, there are a number of documents related to the matter of present invention; however, all of them show disadvantages such as: effects and levels of therapeutic effect duration are lower than expected; some of them are limited to small animal species such as dogs, cats and some birds; another more is targeted for use in humans and other disadvantages.

### BRIEF DESCRIPTION OF THE INVENTION

Present invention refers to an ivermectin emulgel pharmaceutical composition combining the incorporation of an aqueous phase including an agent with bioadhesive properties and another with promoter properties, wherein said aqueous phase is dispersed into the oily phase where the active principle is dissolved, thus obtaining a system reflecting a controlled release.

According to above matter, the pharmaceutical composition described in present invention is a very important alternative since it conjugates the following effects:

a) Bioadhesion: Helping to reduce motility and reproduction of infesting parasites, increasing the period of time of the product on the skin of animals;

b) Absorption promoter; It has been demonstrated that by introducing an absorption promoter agent, the active substance skin permeability is increased since a higher drug permeability may be obtained attacking parasites that stay in the host; and,
c) Controlled release: Since it is a two-phase system (oil/water), active principle release depends on its diffusion through the oily phase that includes the gel, whereby such feature is used as an alternative for antiparasitic treatment for small and large animal species.

According to that above, the “pour-on” emulgel pharmaceutical composition comprises: at least an antiparasitic active principle in charge of providing the pharmacological therapeutic effect; an agent with bioadhesive properties helping to reduce motility and reproduction of infesting parasites, increasing the period of time of the product on the skin of animals; an agent with permeation or absorption promoting properties coadjuvant to increase active principle skin permeability; a surface-active agent or surfactant, which is adsorbed in the oil-water interphase, and consequently, the molecules of said surface-active agent form a kind of “bridge” between the polar phase (water) and the non-polar phase (oil), thereby making less abrupt the transition between both phases; an oil which will form the oily phase of the emulsion and which will incorporate the active principle; and a neutralizing agent allowing the formulation to be applied, further increasing system viscosity which becomes more transparent.

Moreover, the present invention also relates to a method for obtaining an ivermectin emulgel pharmaceutical composition, comprising the steps of preparation of aqueous and oily phases as well as emulgel preparation.

### OBJECTS OF THE INVENTION

Taking into account the drawbacks of prior art, it is an object of present invention to provide an ivermectin emulgel pharmaceutical composition of very simple development and preparation but highly effective to be used as antiparasitic agent in veterinary medicine.

Another object of present invention is to provide an ivermectin emulgel pharmaceutical composition which may be used as antiparasitic treatment in small and large animal species.

Still another object of present invention is to provide an ivermectin emulgel pharmaceutical composition functioning as a promoter-bioadhesive pharmaceutical system for “pour on” percutaneous application and with controlled release. It is still a further object of present invention to provide an ivermectin emulgel pharmaceutical composition which bioadhesive features allow reducing motility and reproduction of infesting parasites, increasing at the same time the period of time of the product on the skin of animals.

Still a further object of present invention is to provide an ivermectin emulgel pharmaceutical composition, which formulation provides improvements to drug permeability through skin as it incorporates a permeation promoter agent, applied by percutaneous route and together with its bioadhesive properties, an improvement in antiparasitic treatment may be obtained.

A further object of present invention is to provide a method for obtaining the ivermectin emulgel pharmaceutical composition.

### BRIEF DESCRIPTION OF DRAWINGS

Novel aspects which are deemed characteristic of the present invention will be particularly defined in attached claims. However, the invention itself both in its organization and its operation method jointly with other objects and advantages thereof will be better understood in the following detailed description of a particularly preferred embodiment of the present invention when read in connection with attached drawings wherein:

FIG. 1 shows a graph of permeation kinetics of ivermectin in-vitro through bovine skin, wherein said ivermectin is included in the pharmaceutical composition developed according to a particularly preferred embodiment of present invention.

FIG. 2 shows a graph of the bio-adhesive gel with variable amounts of added glycerin.

### DETAILED DESCRIPTION OF THE INVENTION EMBODIMENTS

The ivermectin pharmaceutical composition described in a particularly preferred embodiment of the present invention, differs from the formulations identified in the state of the art and which are currently commercially available in the market given that said emulgel pharmaceutical composition combines the incorporation of an aqueous phase including an agent with bioadhesive properties and another with promoter properties, where said aqueous phase is dispersed in the oily phase where the active principle is dissolved, whereby a system reflecting a controlled release will be obtained.

Accordingly, the pharmaceutical composition described and claimed in present invention is a very important alternative as it provides the following effects:

a) Bioadhesion: Helping to reduce motility and reproduction of infesting parasites, increasing the period of time of the product on the skin of animals;

b) Absorption promoter; It has been demonstrated that active substance skin permeability is increased by introducing an absorption promoter agent, since higher drug permeability may be obtained attacking the parasites staying in the host; and,
c) Controlled release: Since it is a two-phase system (oil/water), active principle release depends on diffusion through an oily phase that includes the gel, therefore said feature is used as alternative for antiparasitic treatment of small and large animal species.

The semisolid or emulgel “pour-on” pharmaceutical composition while being stable is quite useful in veterinary medicine since it brings together the advantages of two pharmaceutical forms (gel and emulsion) in a single one, providing an improvement in therapeutic treatment and prophylaxis of parasite-caused infections. Said pharmaceutical composition does not demand specialized personnel for topical application in single dosage by incorporating a permeation promoter agent in its formulation.

In accordance with above description, the emulgel “pour-on” pharmaceutical composition described in a particularly preferred embodiment of present invention comprises: at least an antiparasitic active principle in charge of providing the pharmacological therapeutic effect; an agent with bioadhesive properties helping to reduce motility and reproduction of infesting parasites, increasing the period of time of the product on the skin of animals; an agent with permeation or absorption promoting properties coadjuvant to increase active principle skin permeability; a surface-active agent or surfactant, which is adsorbed in oil-water interphase, and because of that, the molecules of said surface-active agent form a kind of “bridge” between the polar phase (water) and the non-polar phase (oil), thereby making less abrupt the transition between both phases; an oil which will form the oily phase of the emulsion and which will incorporate the active principle; and a neutralizing agent which function is to put the system close to neutrality in order to make the formulation applicable, further increasing system viscosity by deprotonizing Carbocol carboxyl groups (COOH) and passing to COO— thus viscosity is increased and the system becomes more transparent.

The at least an antiparasitic active principle is ivermectin and is present in the “pour-on” emulgel pharmaceutical composition in a concentration range from 0.2 to 1% w/w, preferably 0.5% w/w.

The agent with bioadhesive properties is present in the “pour-on” emulgel pharmaceutical composition in a concentration range from 5 to 10% w/w, preferably 8.26% w/w, wherein said agent may be one of the classic hydrocolloid forms of multiple and varied technological use, which are selected from the group comprising: tragacanth gum (high concentration), guar gum, karaya gum (high concentration), sodium alginate, gelatin, chitosan; cellulose derivatives such as methylcellulose (low molecular weight), sodium carboxymethylcellulose (high molecular weight), hydroxyethyl cellulose, hydroxypropylcellulose, polyethylene glycols (high molecular weight), polyvinyl alcohol, Carbopol 940, acrylic and methacrylic acid polymers and copolymers, polyalkylcyanoacrylates, polycarbophil, or any other substance with bioadhesive properties; preferably selecting Carbopol 940 as bioadhesive agent.

The main advantage of adhesive systems is the possibility to increase residence time in-situ, which reduces the number of applications which ideally leads to system retention on the biological surface where active ingredient may be released for absorption with a subsequent increase in bioavailability.

A number of events are required so that bioadhesion may be present, namely: i) an intimate contact between bioadhesive and receptive tissue is given, wherein said contact requires good wettability to allow polymeric chain relaxation; ii) bioadhesive agent penetration with the tissue is present, thus weak chemical bonds and/or physicochemical interactions are apparent.

The agent with permeation or absorption promoting properties is present in the “pour-on” emulgel pharmaceutical composition in a concentration range from 0.05 to 15% v/v, preferably 10% v/v; where said agent is selected among surfactants or surface-active agents, organic solvents, unsaturated fatty acids and some organic materials. Most effective promoter agents are non-ionic surfactants, preferably sorbitol-derived fatty acids, and more preferably sorbitan laureate or instead, organic solvents having a HLB value (acronym for Hydrophilic-Lipophilic Balance) between 6 and 30, which are selected from the group comprising: glycerol ester chemicals, polyglycerol esters, alkyl fatty acid esters, ethoxylated sorbitan esters, alcohol ethoxylates, lanolin ethoxylates, ethoxylated fatty methyl esters and alkanolamides, or any other substance with absorption promoting properties on skin, preferably selecting Transcutol®.

An adsorption or permeation promoter agent is incorporated since the skin opposes natural resistance to passage of exogenous substances, therefore being necessary to include substances which increase partition and diffusion of lipophilic or hydrophilic substances towards or through the permeability barrier in topical or transdermal formulations, said promoters acting also as co-surfactants in emulgel. Co-surfactants are generally used in microemulsions and their role is to decrease surface tension and to have a small negative value wherein the interphase could be expanded to form fine dispersed droplets and subsequently adsorbing more surfactant and co-surfactant until the concentration volume is quite depleted to return to a positive interfacial tension value.

The surface-active agent or surfactant is present in the “pour-on” emulgel pharmaceutical composition in a concentration range from 0.05 to 15% v/v, preferably between 0.1 and 10% v/v, where said surfactant is selected from the group comprising Pluronic F68, Tween® 60, Tween® 80, Span® 60, Span® 80; preferably using Pluronic F68.

Oil is present in the “pour-on” emulgel pharmaceutical composition in a concentration range from 11.2 to 55.5% w/v, preferably 27.6% w/v; wherein said oil is selected preferably from medium chain triglycerides, being plant-origin fats especially obtained from coconut and palm oil, preferably using Captex® 200.

Selected substance was medium chain triglycerides, since such compound shows very particular properties above other types of oils, showing great compatibility with skin, having cutaneous tolerance. This kind of substance is widely used for manufacturing semisolid systems such as emulsions and ointments and being further the necessary amount for solubilization of active principle amount present in the formulation.

The neutralizing agent is present in the “pour-on” emulgel pharmaceutical composition in a concentration range from 0.1 to 2.0% v/v, preferably from 0.1 to 1% v/v; using any strong or mild alkali or base, selected from the group comprising sodium hydroxide, potassium hydroxide, ammonium hydroxide, borax, monoethanolamine, diethanolamine, triethanolamine; preferably using triethanolamine as neutralizing agent.

As mentioned before, one of the novel features of the pharmaceutical composition of the present invention is its pharmaceutical form which is an emulgel, wherein the gel is incorporated into the aqueous phase, while the active principle is incorporated into the oily phase. In this context, the present invention also provides the method to obtain the ivermectin emulgel pharmaceutical composition comprising the steps of:

I. Aqueous Phase (FA)+Gel:

(a) Placing an amount in the range from 5 to 10 g, preferably 8.26 g of bioadhesive agent, preferably Carbopol 940, in 100 ml of distillate water and leaving moisturizing for 24 hours;

b) Homogenizing with a variable speed stirrer with a serrated propeller in order to form a gel without air;

(c) Adding to above gel solution an amount in the range from 50 to 65 ml, preferably 58.68 ml of glycerin and mixing with a variable speed stirrer with marine propeller until full component homogenization, obtaining preferably a 10% bioadhesive gel. In order to increase skin/solvent partition coefficient, water transport from skin to oil solution might influence active principle permeation, thus being said that substances which increase hydration promote in most cases active absorption, moisturizers such as glycerin may absorb water from skin increasing water content in stratum corneum and therefore decreasing their resistance;

(d) Weighing a required amount of solution obtained in above step (c), wherein said amount will be in function of the amount of product intended to prepare, i.e., if 100 g of product were prepared then the gel amount (cbp) would be 55.8 g; and adding an amount in the range from 2 to 10 ml, preferably 5 ml of surface-active agent, preferably Pluronic F68, which must be previously dissolved in a minimum amount of distillate water, heating at a temperature of 45° C. and homogenizing with the variable speed stirrer and marine propeller. According to its solubility properties, poloxamer is very soluble in water therefore the minimum sufficient amount would be 1 or 2 ml of water to be solubilized, wherein said water volume would be negligible within the formulation.

(e) Adding slowly and with constant stirring to above mixture, an amount in the range from 3 to 8 ml, preferably 5 ml, of permeation promoter agent, preferably Transcutol®, keeping stirring and a temperature of 45° C.; and, (f) Adding to the solution obtained in above step (e) an amount in the range from 0.5 to 2.0 ml, preferably 1.1 ml of neutralizing agent, preferably triethanolamine, by a dropwise addition and constantly stirring to finally get an opalescent gel.

II. Oily Phase (FO):

(g) Dissolve an amount in the range from 0.2 to 1 g, preferably 0.5 g of active principle, preferably ivermectin, from 25 to 30 ml, preferably 27.6 ml of an oil, preferably medium chain triglycerides, and more preferably Captex® 200, keeping a temperature of 45° C. and constantly stirring with a magnetic bar until complete dissolution, within a period from 30 to 45 minutes;

III. Preparation of an Emulgel:

(h) Constantly stirring the aqueous phase obtained in steps (a) to (f) and adding slowly the oily phase obtained in step (g) until complete incorporation, wherein both phases must be at a temperature between 40 and 50° C., preferably at 40° C., and continuing stirring to room temperature.

The present invention will be better understood from the examples described below which are only for illustrative but not limitative purposes, allowing a full understanding of the embodiments of the present invention, this without implying that there are not any other embodiments which were not herein disclosed and which may be put into practice based on the detailed description of such embodiments in the description of the present invention.

### Example 1

I. Preparation of Aqueous Phase (FA):

8.26 g of Carbopol 940 were poured in 100 ml de distillate water and moisturized for 24 hours. Then, it was homogenized after said time with a variable speed stirrer with a serrated propeller in order to form a gel without air. An amount of 58.68 ml of glycerin was immediately added to above gel solution and mixed with a variable speed stirrer with marine propeller until full component homogenization, obtaining a 10% bioadhesive gel.

55.8 g of obtained bioadhesive gel were weighed and 5 ml Pluronic F68 as surface-active agent were added, which was previously dissolved in a minimum amount of distillate water, and the solution was heated at a temperature of 45° C. homogenized with the variable speed stirrer and marine propeller. 5 ml of Transcutol® as permeation promoter agent was slowly added and with constant stirring to above mixture, keeping stirring and a temperature of 45° C.

Finally, 1.1 ml of triethanolamine as neutralizing agent was added to the obtained solution, by a dropwise addition and constantly stirring to obtain an opalescent gel.

II. Preparation of Oily Phase (FO):

0.5 g of ivermectin as active principle was dissolved in 27.6 ml of Captex® 200, keeping a temperature of 45° C. and constantly stirring with a magnetic bar until complete dissolution, taking a time between 30 and 45 minutes.

III. Preparation of Emulgel:

The oily phase was slowly added to the aqueous phase constantly stirring until its full incorporation, wherein both phases were at a temperature of 40° C., and continued stirring until reaching room temperature to obtain finally an emulgel.

### Example 2

TABLE 1
Combination of an oily-phase and surfactant
for emulgel preparation

| SYSTEM | COMPONENTS | % SURFACTANTS |
|---|---|---|
| 1 | IPM-Tween ® 80 | 5 |
| 2 | IPM-Tween ® 60 and Span ® 60 | 5 |
| 3 | IPM-Tween ® 80 and Span ® 80 | 5 |
| 4 | IPM-Tween ® 60 and Span ® 60 | 10 |
| 5 | IPM-Pluronic ® F-68 | 5 |
| 6 | TCM-Tween ® 80 and Span ® 80 | 5 |
| 7 | TCM-Tween ® 80 and Span ® 60 | 5 |
| 8 | TCM-Pluronic ® F68 | 5 |
| 9 | TCM-Plronic ® F68 | 7 |
| 10 | TCM-PemulenTR-1(self | 0.4 |
| | emulsifiers) | |
| 11 | TCM-Pemulen TR-2(self | 0.1 |
| | emulsifiers) | |
| 12 | TCM-Pluronic ® | 5 and 10 |
| | F68/Transcutol | respectively |

The following procedure was carried out for emulgel preparation of 1, 2, 3, 4, 6 and 7 systems:

a) Oily phase: Active ingredient (ivermectin) was dissolved in medium chain triglycerides (TCM) or isopropyl myristate (IPM) as oily phase, according to the prepared system (see table 1), stirred with a magnetic bar at a temperature of 45° C. until dissolution (30 to 45 minutes). Surfactant (Span® 80 or 60) was added at the same temperature, homogenizing with a magnetic bar.

b) Aqueous phase: 10 g of Carbopol 940 were placed in 100 ml of distillate water and moisturized during 24 hours. It was then homogenized with a variable speed stirrer with a serrated propeller in order to form a gel without air.

Subsequently, Carbopol 940 gel, glycerin and distillate water were mixed in a beaker, mixed in a variable speed stirrer with marine propeller until component homogenization.

The required amount of this solution was weighed and surfactant (Tween® 80 or 60) was added, homogenizing the solution and heating at 45° C. TEA was slowly added with stirring for emulgel neutralization.

c) Emulgel formation: Aqueous phase was kept under stirring and the oily phase was added slowly until complete incorporation (both phases were at 40° C.). Stirring continued up to room temperature.

For emulgel system preparation (5, 8, 9, 10, 11 and 12), the same procedure described above was carried out and only varying the oily phase or surfactant, depending on the prepared system (see table 1). For emulgel systems 10 and 11 surfactant was directly added without previous dissolution as it shows self emulsifiable properties.

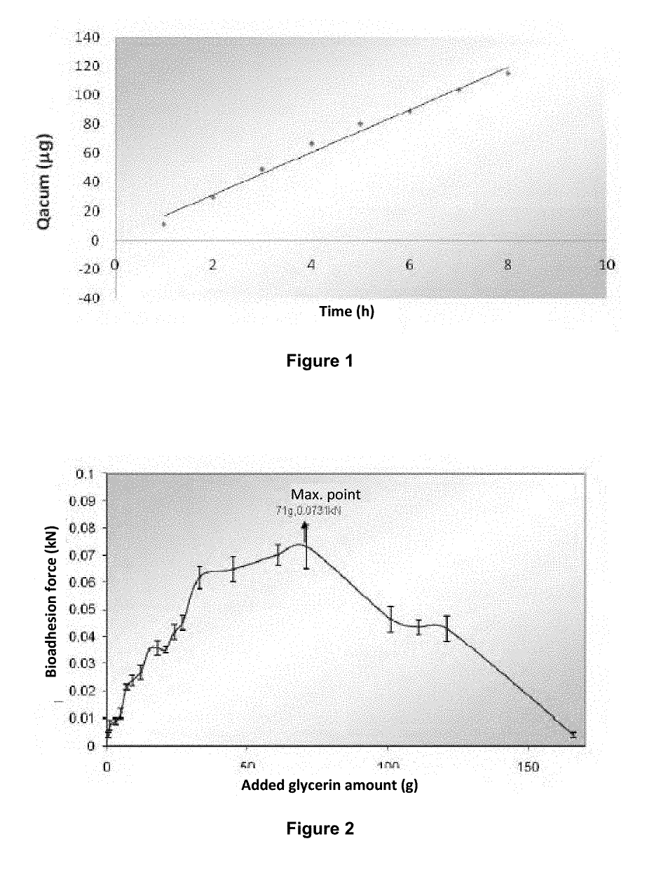

FIG. 1 in attached drawings shows the results of permeation kinetics obtained for emulgel, said test was performed with Franz-type diffusion cells, where a 1.5 cm² sample of bovine skin between donor compartment and receiver compartment was placed, which simulates animal skin to be treated. A formulation sample of about 5 g was placed in the donor compartment, equivalent to 125 mg ivermectin, while a 3% Brij 58 solution was placed in the receiver compartment simulating the receiver environment in body and keeping snick conditions. Samples of 19 ml of reception medium were withdrawn from said system, replacing with fresh medium and making a correction by dilution effect, said samples were taken at time intervals of 1, 2, 3, 4, 5, 6, 7 and 8 hours and permeated ivermectin amount was quantified. A determination of retained drug with skin used as membrane was carried out to quantify the amount of permeated drug. Said test demonstrates that an increase in permeated drug is achieved by having a permeation promoter agent included in the formulation.

TABLE 2
Results from bioadhesion force for different gel
concentrations.

| Polymer | | | | | |
|---|---|---|---|---|---|
| Concentration | Bioadhesion | | | | |
| (%) | Force (kN) | | | Average | δ (+/−) |
| 10 | 0.126 | 0.121 | 0.133 | 0.127 | 0.006 |
| 7 | 0.098 | 0.101 | 0.105 | 0.101 | 0.003 |
| 5 | 0.076 | 0.085 | 0.083 | 0.081 | 0.004 |
| 3 | 0.071 | 0.080 | 0.078 | 0.076 | 0.004 |

Table 2 shows that there is a directly proportional relationship between polymer concentration used for gel preparation and bioadhesion since by increasing the amount of polymer in gel there is a higher number of hydrated polymeric chains and therefore bioadhesion is higher. Results in table also indicate that there is not any significant difference among obtained values. Thus, bioadhesive agent (Carbopol 940) concentration was influenced by system viscosity, selecting the gel at 8.26% polymer concentration.

Upon increasing the molecular weight of polymers or their concentration, the viscosity is also increased. The gel with a 8.26% Carbopol 940 concentration in water was chosen since a system with a viscosity capable of having several features was intended to obtain, such as; easy and practical administration, high handling capacity during formulation.

Viscosities with polymer concentrations higher than 10% (w/v) in water, do not allow gel handling for potential application as “pour on” system.

The present invention also relates to the use of the pharmaceutical composition comprising ivermectin emulgel for manufacturing a medicament for parasitic disease treatment.

Results of Application of 0.5% Ivermectin-Based Emulgel Pharmaceutical Composition in Dogs with Induced Anclyostoma caninum Infection

The following methodology was approved by the Ethical Committee of FES-Cuautitlan (CUCEI) and in accordance with the international standards on animal care and use in experimental procedures. Animals were fed with pelletized commercial feed and water ad libitum. An oral infection was induced by Anclyostoma caninum to three 4-month age dogs and with an approximate weight of 12 kg with a single dose of 200 active larvae.

Three fecal samplings were made before treatment (days 1, 3 and 4) determining the number of eggs per gram of sample (h/g) by McMaster quantitative technique. The therapeutic treatment consisted of applying on day 4 a sufficient amount of 0.5% ivermectin emulgel to cover an area of 50 cm² through a thin and homogeneous film (10×5 cm preformed mold) on dog's back analyzing the daily number of eggs per gram from day 5 (24 hours after treatment) to 9. Animals were later slaughtered making the corresponding necropsy to determine the contents of parasites present in TGI. Results are summarized in following Table 3:

TABLE 3
Results on the number of Anclyostoma caninum eggs per gram of feces
(h/g) for dogs treated on day 4 with 0.5% ivermectin emulgel.

| | Days | | | | | | | | |
|---|---|---|---|---|---|---|---|---|---|
| Dog | 1 | 3 | 4 | 5 | 6 | 7 | 8 | 9 | Necropsy |
| A | 1700 | 1500 | 1200 | Negative | Negative | Negative | Negative | Negative | Negative |
| | h/g | h/g | h/g | | | | | | |
| B | 800 | 650 | 250 | Negative | Negative | Negative | Negative | Negative | Negative |
| | h/g | h/g | h/g | | | | | | |
| C | 3050 | 3150 | 450 | Negative | Negative | Negative | Negative | Negative | Negative |
| | h/g | h/g | h/g | | | | | | |

The results show that even when eggs were not detected from day 5, i.e., 24 hours after treatment, necropsy reported that animals were completely free of parasites. These findings clearly show the efficacy of ivermectin emulgel pharmaceutical composition of present invention and its capacity to provide therapeutically effective plasma levels.

Even though above description refers to certain embodiments of the ivermectin emulgel pharmaceutical composition of present invention, it should be emphasized that several modifications to said embodiments are possible but without separating from the true scope of the invention, such as modifying the type and concentration of the therapeutic agent, bioadhesive, oil and permeability promoter agent and other related modifications but without being far from the true scope of said present invention. Therefore, the present invention shall not be restricted except for the provisions in the state of the art and in the attached claims.

1. A pharmaceutical composition comprising: at least an antiparasitic active principle in charge of providing a pharmacological therapeutic effect; an agent with bioadhesive properties helping to reduce motility and reproduction of infesting parasites, increasing the period of time of the product on the skin of animals; an agent with permeation or absorption promoting properties coadjuvant to increase active principle skin permeability; a surface-active agent or surfactant, which is adsorbed in an oil-water interphase, and consequently, the molecules of said surface-active agent form a bridge between the polar phase (water) and the non-polar phase (oil), thereby making less abrupt the transition between both phases; an oil which will form the oily phase of the emulsion and which will incorporate the active principle; and a neutralizing agent which function is to put the system close to neutrality in order to make the formulation applicable, further increasing system viscosity by deprotonizing Carbocol carboxyl groups (COOH) and passing to COO— whereby viscosity is increased and the system becomes more transparent.

2. The pharmaceutical composition according to claim 1, wherein the pharmaceutical composition is present in semisolid or pour on emulgel form, wherein the gel is incorporated in an aqueous phase, while the active principle is incorporated in an oily phase.

3. The pharmaceutical composition according to claim 2, wherein the at least one antiparasitic active principle is ivermectin.

4. The pharmaceutical composition according to claim 3, wherein ivermectin is present in the composition in a concentration range from 0.2 to 1% w/w.

5. The pharmaceutical composition according to claim 4, wherein ivermectin is present in the composition in a concentration of 0.5% w/w.

6. The pharmaceutical composition according to claim 1, wherein the agent with bioadhesive properties is selected from the group comprising: tragacanth gum, guar gum, karaya gum, sodium alginate, gelatin, chitosan; cellulose derivatives such as methylcellulose, sodium carboxymethylcellulose, hydroxyethyl cellulose, hydroxypropylcellulose, polyethylene glycols polyvinyl alcohol, Carbopol 940, acrylic and methacrylic acid polymers and copolymers, polyalkylcyanoacrylates, polycarbophil, or any other substance with bioadhesive properties.

7. The pharmaceutical composition according to claim 6, wherein the agent with bioadhesive properties is Carbopol 940.

8. The pharmaceutical composition according to claim 7, wherein the agent with bioadhesive properties is present in the pharmaceutical composition in a concentration range from 5 to 10% w/w.

9. The pharmaceutical composition according to claim 8, wherein the agent with bioadhesive properties is present in the pharmaceutical composition in a concentration of 8.26% w/w.

10. The pharmaceutical composition according to claim 1, wherein the agent with permeation or absorption promoting properties is selected from surface-active agents or surfactants, organic solvents, unsaturated fatty acids and some organic materials, wherein the more effective promoter agents are non-ionic surfactants, such as sorbitol-derived fatty acids, and more preferably sorbitan laureate or instead, organic solvents having a HLB value Hydrophilic-Lipophilic Balance value) between 6 and 30, which are selected from the group comprising: glycerol ester chemicals, polyglycerol esters, alkyl fatty acid esters, ethoxylated sorbitan esters, alcohol ethoxylates, lanolin ethoxylates, ethoxylated fatty methyl esters and alkanolamides, or any other substance with absorption promoting properties on skin.

11. The pharmaceutical composition according to claim 10, wherein the agent with permeation or absorption promoting properties is Transcutol®.

12. The pharmaceutical composition according to claim 11, wherein the agent with permeation or absorption promoting properties is present in the pharmaceutical composition in a concentration range from 0.05 to 15% v/v.

13. The pharmaceutical composition according to claim 12, wherein the agent with permeation or absorption promoting properties is present in the composition in a concentration of 10% v/v.

14. The pharmaceutical composition according to claim 1, wherein the surface-active agent or surfactant is selected from the group comprising Pluronic F68, Tween® 60, Tween® 80, Span® 60, Span® 80.

15. The pharmaceutical composition according to claim 14, wherein the surface-active agent or surfactant is Pluronic F68.

16. The pharmaceutical composition according to claim 15, wherein the surface-active agent or surfactant is present in the pharmaceutical composition in a concentration range from 0.05 to 15% v/v.

17. The pharmaceutical composition according to claim 16, wherein the surface-active agent or surfactant is present in the pharmaceutical composition in a concentration between 0.1 and 10% v/v.

18. The pharmaceutical composition according to claim 1, wherein the oil is selected from medium chain triglycerides, which are plant-origin fats especially obtained from coconut and palm oil.

19. The pharmaceutical composition according to claim 18, wherein the oil is Captex® Captex 200.

20. The pharmaceutical composition according to claim 19, wherein the oil is present in the composition in a concentration range from 11.2 to 55.5% w/v.

21. The pharmaceutical composition according to claim 20, wherein the oil is present in the composition in a concentration of 27.6% w/v.

22. The pharmaceutical composition according to claim 1, wherein any strong or mild alkali or base is used as neutralizing agent selected from the group comprising sodium hydroxide, potassium hydroxide, ammonium hydroxide, borax, monoethanolamine, diethanolamine, triethanolamine.

23. The pharmaceutical composition according to claim 22, wherein the neutralizing agent is triethanolamine.

24. The pharmaceutical composition according to claim 23, wherein the neutralizing agent is present in the composition in a concentration range from 0.1 to 2.0% v/v.

25. The pharmaceutical composition according to claim 24, wherein the neutralizing agent is present in the composition in a concentration range from 0.1 to 1% v/v.

26. The use of a pharmaceutical composition comprising ivermectin of claim 1, in manufacturing a medicament for treatment of parasitic diseases.

27. A method of preparation of the pharmaceutical composition of claim 1, characterized by comprising the steps of:
(a) placing an amount in the range from 5 to 10 g of bioadhesive agent in 100 ml de distillate water and moisturizing for 24 hours;
(b) homogenizing with a variable speed stirrer with a serrated propeller in order to form a gel without air;
(c) adding to above gel solution an amount in the range from 50 to 65 ml of glycerin and mixing with a variable speed stirrer with marine propeller until full component homogenization, obtaining a bioadhesive gel preferably at 10%;
(d) weighing a required amount of solution obtained in above step (c), wherein said amount will be in function of the amount of product intended to prepare and adding an amount in the range from 2 to 10 ml of surface-active agent, which must be previously dissolved in a minimum amount of distillate water, heating at a temperature of 45° C. and homogenizing with the variable speed stirrer and marine propeller;
(e) adding slowly and with constant stirring to above mixture, an amount in the range from 3 to 8 ml of permeation promoter agent, keeping stirring and a temperature of 45° C.;
(f) adding to the solution obtained in above step (e) an amount in the range from 0.5 to 2.0 ml, preferably 1.1 ml, del neutralizing agent, preferably triethanolamine, by dropwise addition and constantly stirring to finally obtain the aqueous phase obtaining an opalescent gel.
(g) dissolving an amount in the range from 0.2 to 1 g of active principle in 25 to 30 ml of oil, keeping a temperature of 45° C. and constantly stirring with a magnetic bar until complete dissolution, during a time from 30 to 45 minutes, obtaining the oily phase;
(h) constantly stirring the aqueous phase obtained in steps (a) to (f) and adding slowly the oily phase obtained in step (g) until complete incorporation, wherein both phases must be at a temperature of between 40 and 50° C., and continuing stirring to room temperature.
